(19) United States
(12) Patent Application Publication
(10) Pub. No.: US 2006/0165779 A1
(43) Pub. Date: Jul. 27, 2006

(54) Novel method stabilizing bupropion hydrochloride tablets

(76) Inventors: Manish Chawla, Rubini (IN); Rajeev Singh Raghuvanshi, New Delhi (IN); Ashok Rampal, Amritsar (IN)

Correspondence Address: RANBAXY INC., 600 COLLEGE ROAD EAST, SUITE 2100, PRINCETON, NJ, 08540, US

(21) Appl. No.: 10/511,486

(22) PCT Filed: Apr. 15, 2003
(86) PCT No.: PCT/IB03/01416

§ 371 Date: May 27, 2005

(30) Foreign Application Priority Data
Apr. 15, 2002 (IN) 459/DEL/2002

(51) Int. Cl. A61K 9/20; A61K 31/195

(52) U.S. Cl. 424/464; 514/563

(57) ABSTRACT

The present invention relates to a stable bupropion hydrochloride tablet and a method of stabilizing bupropion hydrochloride tablets, which also serves as an improved tabletting process for the preparation of sustained release bupropion hydrochloride tablets.

### TECHNICAL FIELD OF THE INVENTION

The present invention relates to a stable bupropion hydrochloride tablet and a method of stabilizing bupropion hydrochloride tablets, which also serves as an improved tabletting process for the preparation of sustained release bupropion hydrochloride tablets.

### BACKGROUND OF THE INVENTION

Bupropion hydrochloride is a well-known antidepressant and a non-nicotine aid to smoking cessation. GLAXOSMITHKLINE sells it in United States as WELLBUTRIN® (bupropion hydrochloride immediate release tablets), WELLBUTRIN® SR and ZYBAN® SR (bupropion hydrochloride sustained release tablets). Bupropion hydrochloride also has utility as an anticholesterol agent, in suppressing prolactin secretion, in preventing functional impairment and drowsiness seen upon administration of benzodiazepine, in the treatment of minimal brain dysfunction, tardive dyskinesia, impaired mental alertness upon ingestion of ethanol and psychosexual dysfunction.

Bupropion hydrochloride is a water-soluble, crystalline solid, which is highly hygroscopic and susceptible to decomposition. Because of the drug's instability, researchers working in this field have tried a number of different approaches to improve the storage stability of the drug in the formulation. U.S. Pat. Nos. 5,358,970; 5,763,493; 5,731,000; 5,427,798; 5,968,553; 5,541,231; and 6,242,496 variously disclose the use of organic acids, carboxylic acids, dicarboxylic acids, inorganic acids, acid salts of an amino acids, sodium metabisulfite, and sodium bisulfate as stabilizers for bupropion compositions. A potential disadvantage of using acidic materials in pharmaceutical formulations, such as those disclosed above, is the possible need to provide costly production procedures and equipment.

For example, U.S. Pat. No. 5,358,970, which is incorporated herein in its entirety by reference, states that suitable stabilizers are those which have an aqueous solution pH of about 0.9 to about 4 at an aqueous solution concentration of about 6% w/w and are a solid or liquid at 30° C. The '970 patent further states that specific suitable stabilizers that meet the pH range and are therefore useful include: L-cysteine hydrochloride, glycine hydrochloride, ascorbic acid, malic acid, sodium metabisulfite, isoascorbic acid, citric acid, tartaric acid, L-cystine dihydrochloride. L-cysteine hydrochloride and glycine hydrochloride.

U.S. Pat. No. 5,763,493, which is incorporated herein in its entirety by reference, states the stabilizer is selected from an organic acid, a carboxylic acid other than ascorbic acid and isoascorbic acid, an acid salt of an amino acid, and sodium metabisulphite, and further states that the preferred pH of the aqueous solution of the stabilizer is 0.9 to about 2 and most preferably 1.

U.S. Pat. No. 5,731,000, which is incorporated herein in its entirety by reference, describes suitable stabilizers as including organic acids, carboxylic acids, acid salts of amino acids and sodium metabisulphite and further states that preferably, the acid salts of amino acids are hydrochloride salts such as cysteine hydrochloride, glycine hydrochloride or cystine dihydrochloride. Other preferred examples of stabilizers are described as including ascorbic acid, malic acid, isoascorbic acid, citric acid and tartaric acid, and that L-cysteine hydrochloride and glycine hydrochloride are the most preferred stabilizers.

U.S. Pat. No. 5,968,553, which is incorporated herein in its entirety by reference, characterizes suitable stabilizers as being inorganic acids having an aqueous solution pH of from about 0.5 to about 4.0 at a concentration of about 0.31% w/w. The '553 patent states that suitable stabilizers include inorganic acids meeting the above criteria and include hydrochloric acid, phosphoric acid, nitric acid, and sulfuric acid, or combinations thereof. Hydrochloric acid is described as being a preferred stabilizer.

U.S. Pat. No. 6,242,496, which is incorporated herein in its entirety by reference, describes the stabilizers as including dicarboxylic acids including oxalic, succinic, adipic, fumaric and phthalic acids, or combinations thereof, and that fumaric acid is a preferred stabilizer.

U.S. Pat. No. 5,427,798 describes formulations in which drug release is achieved in a controlled manner by varying the surface area to volume ratio of the tablet. However, U.S. Pat. No. 5,427,798 relies on the inclusion of acids to stabilize the bupropion hydrochloride.

U.S. Pat. No. 6,306,436 discloses stabilized bupropion hydrochloride pharmaceutical compositions that are free of added acid and provide for sustained release of bupropion hydrochloride. Stabilization is achieved by using particulate bupropion hydrochloride, which is coated with a membrane coating or by large size bupropion crystals. Although avoiding the potential disadvantages of using an acid, a potential disadvantage of using the disclosure of U.S. Pat. No. 6,306,436 is that a drug particle coating may be an expensive and time-consuming process.

U.S. Pat. No. 6,238,697 describes methods and formulations for making extended release bupropion hydrochloride tablets using a direct compression method. In the disclosed methods and formulations, tablets are formed that combine bupropion hydrochloride, binders, fillers, glidants and lubricants and processing under low shear conditions that result in hard, chip-resistant tablets that exhibit improved cohesiveness and are easily and reproducibly formed without adhering to the compression punches. The disclosed methods and formulations employ the use of sodium sulfite or potassium metabisulfite to improve the stability of bupropion hydrochloride.

Direct compression requires the use of specific excipients of particular size and density to avoid the problems of segregation and non-uniform content of the drug product. Requiring a process to use excipients of a specific particle size and density range, however, adds to costs and makes the process less robust. Moreover, the success of the direct compression process further depends on bulk density, tap density and particle size distribution of the drug.

Most of the prior art researchers have used wet granulation methods to prepare bupropion hydrochloride immediate release or sustained release tablets.

Wet granulation provides better content uniformity, but is not advisable for active ingredients, such as bupropion hydrochloride, that are hygroscopic and susceptible to decomposition. Moreover, polymers, especially the hydrophilic polymers typically usually used in achieving extended release, interact with the aqueous system making wet granulation a cumbersome process. The wet granulation process with hydrophilic polymers may also result in variable release characteristics depending on the degree of hydration of the polymer. Even the fluid volume of the granulating agent and granulation time may also affect the release characteristics. Further, use of an organic solvent in the process leads to the problem of residual solvents and extra cost for maintaining the environmental standards inside the plant and in the outside surroundings.

Hence, there is a need for not only a better stabilization method, but also for an improved tabletting process.

### SUMMARY OF THE INVENTION

In one general aspect there is provided a stable bupropion hydrochloride tablet. The tablet is free of stabilizer and contains at least about 80% of undegraded bupropion hydrochloride after storage for two months at 40° C. and 75% relative humidity.

Embodiments of the tablet may include one or more of the following features. For example, the tablet may be a sustained release tablet. The tablet may include bupropion hydrochloride, one or more release rate controlling polymers, and one or more diluents, binders, lubricants, glidants and coloring agents. The release rate controlling polymers may include one or more of cellulose derivatives, acrylates, polyvinlyacetate/povidone mixtures, polyethylene oxides, starches and their derivatives, gums, alginates, carbohydrate based polymers, polysaccharide, and combinations thereof.

The cellulose derivative may be one or more of ethyl cellulose, methylcellulose, hydroxymethylcellulose, hydroxyethylcellulose, hydroxypropylcellulose, hydroxypropyl methylcellulose, sodium carboxymethylcellulose, and combinations thereof. The cellulose derivative may be hydroxypropyl cellulose. The acrylate may be one or more of carbomer, polycarbophil, and EUDRAGIT®. The carbomer may include one or more of Carbopol®-971 P, 974 P, and 934 P.

The binder may be one or more of starch, gelatin, highly dispersed silica, mannitol, lactose, polyethylene glycol, polyvinylpyrrolidone, cross-linked polyvinylpyrrolidone, cross-linked carboxymethyl cellulose, hydroxypropyl methylcellulose, hydroxypropyl cellulose and natural and synthetic gums. The diluent may be microcrystalline cellulose. The lubricant may be stearic acid.

In another general aspect, there is provided a method of stabilizing bupropion hydrochloride tablets by using a dry granulation process. The dry granulation process includes a) blending bupropion hydrochloride and one or more pharmaceutically acceptable excipient(s), b) compacting or slugging the material of step (a), c) sizing the compacted or slugged material of step (b) into granules, and d) compressing the granules of step (c).

The method may include one or more of the following features. For example, the tablet may contain at least about 80% of undegraded bupropion hydrochloride after storage for two months at 40° C. and 75% relative humidity.

Step (b) may be compaction. The compaction may include using a roller compactor. Step (c) may be milling. The method may further include lubricating the sized granules of step (c) before compressing the granules. The method may still further include coating the tablet after compressing the granules.

The one or more pharmaceutically acceptable excipients may be one or more of release rate controlling polymers, diluents, binders, lubricants, glidants, and coloring agents. The release rate controlling polymers may be one or more of cellulose derivatives, acrylates, polyvinlyacetate/povidone mixtures, polyethylene oxides, starches and their derivatives, gums, alginates, carbohydrate based polymers, polysaccharide, and combinations thereof. The cellulose derivative may be one or more of ethyl cellulose, methylcellulose, hydroxymethylcellulose, hydroxyethylcellulose, hydroxypropylcellulose, hydroxypropyl methylcellulose, sodium carboxymethylcellulose, and combinations thereof. The cellulose derivative may be hydroxypropyl cellulose. The acrylate may be one or more of carbomer, polycarbophil, and EUDRAGIT®. The carbomer may be one or more of Carbopol®-971P, 974P and 934P.

The binder may be one or more of from starch, gelatin, highly dispersed silica, mannitol, lactose, polyethylene glycol, polyvinylpyrrolidone, cross-linked polyvinylpyrrolidone, cross-linked carboxymethyl cellulose, hydroxypropyl methylcellulose, hydroxypropyl cellulose, and natural or synthetic gums. The diluent may be microcrystalline cellulose. The lubricant may be stearic acid. The bupropion hydrochloride tablets may be free of stabilizer.

In another general aspect, a method of one or both of treating depression and providing smoking cessation is provided. The method includes providing bupropion hydrochloride in a dosage form that is free of stabilizer and contains at least about 80% of undegraded bupropion hydrochloride after storage for two months at 40° C. and 75% relative humidity.

Embodiments of the method may include any one or more of the features described above. For example, the dosage form may be produced using a dry granulation process that includes (a) blending bupropion hydrochloride and one or more pharmaceutically acceptable excipients, (b) either compacting or slugging the blend of step (a), sizing the compacted or slugged material of step (b) into granules, and (d) compressing the granules of step (c).

The methods, processes, and formulations described herein may provide one or more of the following features. For example, the method is simple and produces tablets having good stability during storage and desired sustained release characteristics. The method can avoid the use of an acid stabilizer, coated bupropion hydrochloride particles, and larger sized bupropion hydrochloride crystals, thereby resulting in reduced costs. The method can also eliminate the use of organic solvent during wet granulation. Therefore, the problem of residual solvent is nonexistent. The method can also eliminate the variability in the degree of hydration of hydrophilic polymers and its consequent effect on release characteristics.

The method can provide granules with consistent hardness and increased density. Granules for high-speed tabletting or encapsulation are produced with reproducible granule size distribution. Less variation in particle size distribution reduces the need for reprocessing fines. The process can provide a good reprocessing potential as the compacts or slugs and tablets can be crushed into powder and re-compacted to make the tablets without affecting drug release profiles.

The details of one or more embodiments of the invention are set forth in the accompanying drawings and the description below. Other features, objects, and advantages of the invention will be apparent from the description and the claims.

### DETAILED DESCRIPTION OF THE INVENTION

The inventors have discovered that stable bupropion hydrochloride tablets can be prepared by a dry granulation process without having to add any stabilizer. The inventors also have discovered that the process of dry granulation further serves as an improved tabletting process for the preparation of sustained release bupropion hydrochloride tablets. Therefore, the method described herein not only stabilizes bupropion hydrochloride without having to use the acid stabilizer, coated bupropion hydrochloride particles, or larger sized bupropion hydrochloride crystals of the prior art researchers, but also provides a better tabletting process for the preparation of sustained release tablets.

Therefore, one aspect of the present invention is a stable bupropion hydrochloride tablet in which the tablet is free of any stabilizer and contains at least about 80% of undegraded bupropion hydrochloride after storage for two months at 40° C. and 75% relative humidity. Another aspect is a method for preparing a stable bupropion hydrochloride tablet in which the tablet is prepared by a dry granulation process and the tablet contains at least about 80% of undegraded bupropion hydrochloride after storage for two months at 40° C. and 75% relative humidity. Another aspect is a method for preparing a stable sustained release bupropion hydrochloride tablet by a dry granulation process and the tablet contains at least about 80% of undegraded bupropion hydrochloride after storage for two months at 40° C. and 75% relative humidity.

Another aspect is a stable bupropion hydrochloride sustained release tablet that is free of any stabilizer and contains at least about 80% of undegraded bupropion hydrochloride after storage for two months at 40° C. and 75% relative humidity. Examples of stabilizers include sodium sulfite and potassium metabisulfite, as well as acids, such as organic acids, carboxylic acids, dicarboxylic acids, inorganic acids, acid salts of an amino acids, sodium metabisulfite, and sodium bisulfate. As described above, a stabilizer can be characterized by using an aqueous solution pH of about 0.9 to about 4 at an aqueous solution concentration of about 6% w/w and are a solid or liquid at 30° C. Stabilizers that meet that pH range include: L-cysteine hydrochloride, glycine hydrochloride, ascorbic acid, malic acid, sodium metabisulfite, isoascorbic acid, citric acid, tartaric acid, L-cystine dihydrochloride, L-cysteine hydrochloride and glycine hydrochloride. Other stabilizers include organic acids, carboxylic acids, and an acid salt of an amino acid. Acid salts of amino acids include hydrochloride salts such as cysteine hydrochloride, glycine hydrochloride or cystine dihydrochloride. Other stabilizers include dicarboxylic acids including oxalic, succinic, adipic, fumaric and phthalic acids.

The dry granulation process generally includes the steps of:
a) blending bupropion hydrochloride and other pharmaceutically acceptable excipient(s),
b) compacting or slugging,
c) sizing the compacted or slugged material of step (b) into granules, and
d) compressing the granules of step (c) to form tablets.
   Again, the process is free of stabilizers.

The term “bupropion hydrochloride” is used to refer to the hydrochloride salt of m-chloro-α-(t-butylamino)propiophenone.

The pharmaceutically acceptable excipients may be selected from amongst one or more of release rate controlling polymers, diluents, binders, lubricants, glidants, and coloring agents which are compatible with bupropion hydrochloride and which would help in optimizing tablet robustness and drug dissolution from the tablet.

Release rate-controlling polymers may be selected from any pharmaceutically acceptable excipients that can control the rate of release of the active ingredient. For example, such release rate-controlling polymers can be selected from the group that includes cellulose derivatives, acrylates, polyvinlyacetate/povidone mixture, polyethylene oxides, starch and their derivatives, gums, alginates, carbohydrate based polymers, polysaccharides, and combinations thereof.

Cellulose derivative can be selected, for example, from one or more of the group that includes ethyl cellulose, methylcellulose, hydroxymethylcellulose, hydroxyethylcellulose, hydroxypropylcellulose, hydroxypropyl methylcellulose, and sodium carboxymethylcellulose having different degrees of substitution or viscosities and molecular weights. These release rate-controlling polymers can be used alone or in combination. Various degrees of substitution and/or different molecular weights corresponding to a different degree of viscosity can be used as suitable cellulose based rate-controlling polymers.

The term “acrylates” is used to describe linear, non-crosslinked copolymers that contain combinations of acrylic acid, methacrylic acid and their simple esters. Acrylates can be selected from the group that includes carbomer, polycarbophil and EUDRAGIT®.

The name “Carbomer” is used to describe high molecular weight cross-linked homopolymers of acrylic acid. Carbomers commercially available under the trademark Carbopol® may be selected from Carbopol®-934P, 971P or 974P. Methacrylic acid polymers and copolymers commercially available under the trademark EUDRAGIT® s are particularly suitable.

The rate controlling polymer or polymers can be used in a concentration of approximately 5% to approximately 60% of the tablet weight depending on the polymer or polymers used. The use of hydroxypropyl methylcellulose (HPMC), hydroxypropylcellulose, polyvinlyacetate/povidone mixture or Carbopol®-971P is particularly suitable. These polymers swell to form a hydrophilic matrix system, which controls the release of bupropion hydrochloride. The tablet hydrates on wetting with aqueous fluids and the hydrophilic polymers form a gel layer. Due to permeation of aqueous fluid into the tablet the thickness of gel layer is increased, and bupropion hydrochloride diffuses slowly out of the gel layer. Slow erosion of the swollen gel may also contribute to drug release.

Diluents may be selected from one or more of any suitable pharmaceutically acceptable excipient that gives bulk to the composition and improves compressibility. For example, diluents may be selected from the group that includes starch, microcrystalline cellulose, lactose, glucose, mannitol, alginates, alkali earth metal salts, dicalcium phosphate, glyceryl monostearate, and polyethylene glycols. Microcrystalline cellulose is particularly suitable.

Binders may be selected from one or more of any pharmaceutically acceptable excipients that have cohesive properties to act as a binder. For example, suitable excipients include starch, gelatin, highly dispersed silica, mannitol, lactose, polyethylene glycol, polyvinylpyrrolidone, cross-linked polyvinylpyrrolidone, cross-linked carboxymethyl cellulose, hydroxypropyl methylcellulose, hydroxypropyl cellulose, and natural or synthetic gums.

Lubricants may be selected, for example, from one or more of talc, stearic acid, magnesium stearate, other alkali earth metal stearate like calcium, zinc etc., sodium lauryl sulphate, hydrogenated vegetable oil, sodium benzoate, sodium stearyl fumarate, glyceryl monostearate, and PEG 4000.

Glidants may be selected, for example, from colloidal silicon dioxide, talc, and other suitable glidants.

As described above, the ingredients are blended and the blend is compacted by roller compaction. The compactor can have rollers and powder transport screws of different designs. Alternatively, this blend may instead be compressed to make slugs. Whether the blend is compacted or slugged (i.e., compressed to make slugs), either process can be used with bupropion hydrochloride alone or with one or more rate controlling polymers and/or excipient(s).

Next, the compacted or slugged material is sized by a suitable machine, such as an oscillating granulator, Multimill, and/or Fitzmill and sieved into the desired granule size.

As an optional step, the granules that are either too large or too small are recycled and combined with an original powder mix and passed through the roller compactor or tabletting machine. Normally 30-70% of coarse granules (i.e., retained on a 44-mesh sieve and passed through an 18-mesh sieve) are preferred and are usually achieved in a single compaction cycle.

These granules are optionally lubricated with the lubricant and are compressed to form tablets. These tablets optionally may be given a coating to enhance the aesthetic appeal. Optionally, these granules can be capsulated into the hard gelatin capsules.

The following examples are provided to enable one of ordinary skill in the art to prepare dosage forms of the invention and should not be construed as limiting the scope of the invention.

### EXAMPLES 1-4

Bupropion hydrochloride 150-mg formulations

| | Weight (mg) per tablet | | | |
|---|---|---|---|---|
| Ingredient | Example 1 | Example 2 | Example 3 | Example 4 |
| Bupropion hydrochloride | 150.00 | 150.00 | 150.00 | 150.00 |
| Hydroxypropyl cellulose- | 63.00 | — | — | 31.5 |
| M | | | | |
| Polyvinlyacetate/ | — | 63.00 | — | — |
| Povidone mixture | | | | |
| Carbopol ® 971P | — | — | 63.00 | 31.5 |
| Microcrystalline | 200.00 | 200.00 | 200.00 | 200.00 |
| cellulose | | | | |
| Stearic acid | 3.2 | 3.2 | 3.2 | 3.2 |
| Total | 416.00 | 416.00 | 416.00 | 416.00 |

The above bupropion hydrochloride formulations were prepared using the following process:
1. Bupropion Hydrochloride, microcrystalline cellulose, and the rate controlling polymers were sifted through a 44 BSS sieve and lubricated with stearic acid (half of the total quantity),
2. The blend of step 1 was compacted using a roller compacter,
3. The compacts of step 2 were sized through an oscillating granulator and sifted through an 18 BSS sieve.
4. The fines obtained were recycled to achieve the desired ratio of coarse and fines.
5. The granules of step 4 were lubricated with the remaining quantity of stearic acid and compressed into tablets.

The stability of the tablets prepared as per the composition and process of Examples 1-4 at 40° C. and 75% RH is given in Table-1.

TABLE 1
Comparative Stability of Bupropion Hydrochloride Tablets Prepared as
Per the Composition of Examples 1-4 and Commercially Available
Bupropion Hydrochloride Tablets (WELLBUTRIN SR ® tablets).

| | % bupropion hydrochloride | | | | |
|---|---|---|---|---|---|
| | EXAMPLES | | | | |
| Stability conditions | 1 | 2 | 3 | 4 | WELLBUTRIN SR ® |
| Initial | 98.5 | 96 | 102 | 101.8 | 105.3 |
| 1 month at 40° C. | 97.0 | 95 | 101.3 | — | 95.1 |
| and 75% RH | | | | | |
| 2 months at 40° C. | 93.6 | 90.0 | 102.4 | 104.5 | 89.0 |
| and 75% RH | | | | | |

RH = Relative Humidity
*of added quantity

The dissolution profile of the tablets prepared as per the composition and process of Examples 1, 3, and 4 is given in Table-2.

TABLE 2
Dissolution Profiles of Bupropion Hydrochloride (150 mg) Formulations
(in distilled water 900 ml at 50 rpm using USP-2 apparatus).

| Time | % bupropion hydrochloride dissolved | | |
|---|---|---|---|
| (hrs) | Example 1 | Example 3 | Example 4 |
| 0.5 | 22 | 19 | 20 |
| 1 | 34 | 26 | 29 |
| 2 | 48 | 37 | 41 |
| 4 | 64 | 53 | 59 |
| 6 | 73 | 67 | 69 |
| 8 | 78 | 72 | 72 |

The above data in Tables 1 and 2 clearly indicate that the granulation both stabilizes bupropion hydrochloride tablets without any stabilizer and also serves as an improved tabletting process for the preparation of sustained release bupropion hydrochloride tablets.

While several particular forms of the invention have been illustrated and described, it will be apparent that various modifications and combinations of the invention detailed in the text and claims can be made without departing from the spirit and scope of the invention. For example, the dosage formulations described herein can be prescribed for one or more of the following uses: treating depression, providing smoking cessation, as an anticholesterol agent, in suppressing prolactin secretion, in preventing functional impairment and drowsiness seen upon administration of benzodiazepine, in the treatment of minimal brain dysfunction, tardive dyskinesia, impaired mental alertness upon ingestion of ethanol and psychosexual dysfunction. Moreover, it is contemplated that any single feature or any combination of optional features of the inventive variations described herein may be specifically excluded from the claimed invention and be so described as a negative invention. Accordingly, it is not intended that the invention be limited, except as by the appended claims.

We claim:

1. A stable bupropion hydrochloride tablet, wherein the tablet is free of stabilizer and contains at least about 80% of undegraded bupropion hydrochloride after storage for two months at 40° C. and 75% relative humidity.

2. The tablet according to claim 1, wherein the tablet is a sustained release tablet.

3. The tablet according to claim 1, wherein the tablet comprises bupropion hydrochloride, one or more release rate controlling polymers, and one or more diluents, binders, lubricants, glidants and coloring agents.

4. The tablet according to claim 3, wherein the release rate controlling polymers comprises one or more of cellulose derivatives, acrylates, polyvinlyacetate/povidone mixtures, polyethylene oxides, starches and their derivatives, gums, alginates, carbohydrate based polymers, polysaccharide, and combinations thereof.

5. The tablet according to claim 4, wherein the cellulose derivative comprises one or more of ethyl cellulose, methylcellulose, hydroxymethylcellulose, hydroxyethylcellulose, hydroxypropylcellulose, hydroxypropyl methylcellulose, sodium carboxymethylcellulose, and combinations thereof.

6. The tablet according to claim 5, wherein the cellulose derivative comprises hydroxypropyl cellulose.

7. The tablet according to claim 4, wherein the acrylate comprises one or more of carbomer, polycarbophil, and EUDRAGIT®.

8. The tablet according to claim 7, wherein the carbomer comprises one or more of Carbopol®-971 P, 974 P, and 934 P.

9. The tablet according to claim 3, wherein the binder comprises one or more of starch, gelatin, highly dispersed silica, mannitol, lactose, polyethylene glycol, polyvinylpyrrolidone, cross-linked polyvinylpyrrolidone, cross-linked carboxymethyl cellulose, hydroxypropyl methylcellulose, hydroxypropyl cellulose and natural, and synthetic gums.

10. The tablet according to claim 3, wherein the diluent comprises microcrystalline cellulose.

11. The tablet according to claim 3, wherein the lubricant comprises stearic acid.

12. A method of stabilizing bupropion hydrochloride tablets using a dry granulation process, the dry granulation process comprising:
a) blending bupropion hydrochloride and one or more pharmaceutically acceptable excipient(s),
b) compacting or slugging the material of step (a),
c) sizing the compacted or slugged material of step (b) into granules, and
d) compressing the granules of step (c).

13. The method according to claim 12, wherein the tablet contains at least about 80% of undegraded bupropion hydrochloride after storage for two months at 40° C. and 75% relative humidity.

14. The method according to claim 12, wherein step (b) comprises compaction.

15. The method according to claim 14, wherein the compaction comprises using a roller compactor.

16. The method according to claim 12, wherein step (c) comprises milling.

17. The method according to claim 12, further comprising lubricating the sized granules of step (c) before compressing the granules.

18. The method according to claim 12, further comprising coating the tablet after compressing the granules.

19. The method according to claim 12, wherein the one or more pharmaceutically acceptable excipients comprise one or more of release rate controlling polymers, diluents, binders, lubricants, glidants, and coloring agents.

20. The method according to claim 19, wherein the release rate controlling polymers comprise one or more of cellulose derivatives, acrylates, polyvinlyacetate/povidone mixtures, polyethylene oxides, starches and their derivatives, gums, alginates, carbohydrate based polymers, polysaccharide, and combinations thereof.

21. The method according to claim 20, wherein the cellulose derivative comprises one or more of ethyl cellulose, methylcellulose, hydroxymethylcellulose, hydroxyethylcellulose, hydroxypropylcellulose, hydroxypropyl methylcellulose, sodium carboxymethylcellulose, and combinations thereof.

22. The method according to claim 21, wherein the cellulose derivative comprises hydroxypropyl cellulose.

23. The method according to claim 20, wherein the acrylate comprises one or more of carbomer, polycarbophil, and EUDRAGIT®.

24. The method according to claim 23, wherein carbomer comprises one or more of Carbopol®-971 P, 974 P and 934 P.

25. The method according to claim 19, wherein the binder comprises one or more of from starch, gelatin, highly dispersed silica, mannitol, lactose, polyethylene glycol, polyvinylpyrrolidone, cross-linked polyvinylpyrrolidone, cross-linked carboxymethyl cellulose, hydroxypropyl methylcellulose, hydroxypropyl cellulose, and natural or synthetic gums.

26. The method according to claim 19, wherein the diluent comprises microcrystalline cellulose.

27. The method according to claim 19, wherein the lubricant comprises stearic acid.

28. The method according to claim 12, wherein the bupropion hydrochloride tablets are free of stabilizer.

29. A method of one or both of treating depression and providing smoking cessation, the method comprising:
providing bupropion hydrochloride in a dosage form,
wherein the dosage form is free of stabilizer and contains at least about 80% of undegraded bupropion hydrochloride after storage for two months at 40° C. and 75% relative humidity.

30. The method of claim 29, wherein the dosage form is produced using a dry granulation process, the dry granulation process comprising (a) blending bupropion hydrochloride and one or more pharmaceutically acceptable excipients, (b) either compacting or slugging the blend of step (a), sizing the compacted or slugged material of step (b) into granules, and (d) compressing the granules of step (c).
